(19) United States
(12) Patent Application Publication
(10) Pub. No.: US 2024/0094154 A1
(43) Pub. Date: Mar. 21, 2024

(54) SENSOR ASSEMBLY FABRICATION AND SENSOR ASSEMBLY

(71) Applicant: Analog Devices, Inc., Wilmington, MA (US)

(72) Inventors: Alexander C. STANGE, Somerville, MA (US); Mohamed AZIZE, Medford, MA (US); Hari CHAUHAN, Windham, NH (US)

(21) Appl. No.: 18/189,717

(22) Filed: Mar. 24, 2023

Related U.S. Application Data
(63) Continuation-in-part of application No. 17/947,007, filed on Sep. 16, 2022.

(51) Int. Cl. G01N 27/12; B01L 3/00

(52) U.S. Cl. CPC G01N 27/125; B01L 3/502707; B01L 3/502715

(57) ABSTRACT

The present disclosure provides a method of fabricating a sensor assembly in which a sensor surface has an anchor species provided thereon, the anchor species having a first functional group attached. The method further comprises disposing a fluid channel over the surface and subsequently providing an analyte capture species to the fluid channel. The analyte capture species comprises a second functional group configured to react with the first functional group. The surface is then exposed to photo radiation and the first and second functional groups react forming a link between the analyte capture species and the anchor species on the sensing surface.

### CROSS-REFERENCE TO RELATED APPLICATION

This application is a continuation-in-part of U.S. patent application Ser. No. 17/947,007, filed Sep. 16, 2022, the contents of which application are hereby incorporated by reference herein in their entireties.

### FIELD OF THE DISCLOSURE

This disclosure relates to methods for sensor assembly fabrication and sensor assemblies.

### BACKGROUND

Various sensor fabrication methods and sensor assemblies are known. There are many types of sensors that can be fabricated which detect a wide range of analytes, for example biosensors to detect various types of bio-molecule, as well as sensors to detect the presence of inorganic compounds such as nanoparticles and metal ions.

Sensor fabrication methods typically include a step to functionalize a sensing surface (e.g. an electrode) with various analyte capture molecules, followed by incorporation of the sensing surface into a sensor device. However, traditional sensor fabrication methods suffer from drawbacks relating to scale-up inefficiency and there is disruption and/or destruction of the chemical reactivity of functionalized sensing surfaces during processing stages such as encapsulation. For example, the use of high temperatures and the ultrasonic welding can damage functional surfaces.

There are various methods used to achieve this functionalization such as drop-casting molecules in solution onto the sensing surface. The main drawback with drop-casting is the coffee ring effect (CRE) which can occur. The CRE is the formation of a ring-like pattern on evaporation of droplets containing suspended particles onto a surface. The CRE disrupts the distribution of particles on a surface which affects the sensor efficiency and accuracy. Moreover, sensing surfaces are commonly addressed individually with manual or even automated pipettes/nozzles. This is a serial process and may result in inefficiencies when scaling up to hundreds or thousands of sensing areas per wafer/chip.

Following functionalization, the sensing surface can be incorporated into a sensing device. Incorporation can be understood to mean disposing the sensing surface within a layer of a device which is available for exposure to analyte molecules. Incorporation can include the formation of monolayers over the sensing surface (e.g. polymer films) and/or incorporation of sensing surfaces into fluidic channels. The functionalization on the sensing surface can have low chemical stability and be susceptible to degradation/denaturation upon incorporation.

Accordingly, there is a need to provide an improved sensor assembly fabrication method which is efficient and customizable for scale-up and which does not suffer from the drawbacks of chemical instability of functional groups on the sensing surface upon incorporation into a device.

### SUMMARY OF THE DISCLOSURE

The present disclosure provides a method of fabricating a sensor assembly in which a sensor surface has an anchor species provided thereon, the anchor species having a first functional group attached. The method further comprises disposing a fluid channel over the surface and subsequently providing an analyte capture species to the fluid channel. The analyte capture species comprises a second functional group configured to react with the first functional group. The surface is then exposed to photo radiation and the first and second functional groups react forming a link between the analyte capture species and the anchor species on the sensing surface.

In one embodiment, a method of fabricating a sensor assembly comprises the steps of: providing a sensing surface with an anchor species provided thereon, the anchor species comprising a first functional group; disposing a fluid channel over at least a part of the sensing surface such that fluid can be provided to or removed from the sensing surface via the fluid channel; providing an analyte capture species to the fluid channel, wherein each analyte capture species comprises an analyte capture part and a second functional group configured to react with the first functional group; and exposing at least a portion of the sensing surface covered by the fluid channel to photo radiation so as to cause a photo-initiated reaction between the first functional group and the second functional group to thereby couple the analyte capture species to the anchor species on the sensing surface and form a sensing surface with an analyte capture species thereon.

In one embodiment, a sensor assembly is provided is obtained or obtainable using the methods disclosed herein.

In one embodiment, a sensor assembly comprises: a sensing surface with a bridging species provided thereon; an analyte capture species coupled to the sensing surface through the bridging species; and a fluid channel disposed over at least a part of the sensing surface such that fluid can be provided to or removed from the sensing surface via the fluid channel. The bridging species comprises a product of a photo-initiated reaction between a first functional group connected to the sensing surface via an anchor species and a second functional group connected to the analyte capture species.

### BRIEF DESCRIPTION OF THE DRAWINGS

The present invention will now be described in more detail with reference to the accompanying drawings, which are not intended to be limiting:

FIGS. 1A to 1C provide a schematic cross-sectional view of a sensor assembly in a sensor assembly fabrication method according to an embodiment;

FIG. 2 provides a schematic cross-sectional view of a sensor assembly with an analyte bound to an analyte capture species which is coupled to a sensing surface in a sensor assembly fabrication method according to an embodiment;

FIG. 3 provides a schematic cross-sectional view of a sensor assembly with first and second sensing sites on the sensing surface, each with different analytes bound to different analyte capture species coupled to respective sensing sites in a sensor assembly fabrication method according to an embodiment;

FIG. 4 shows panels 4A to 4G that provide a schematic plan view of a sensor assembly with selective photo patterning of the sensing surface in a sensor assembly fabrication method according to an embodiment;

FIG. 5 shows panels 5A and 5B that provide a schematic cross-sectional view of a sensor assembly comprising a target capture surface in sensor assembly fabrication method according to an embodiment;

FIG. 6 provides a block diagram of a sensor assembly fabrication method according to an embodiment.

### DETAILED DESCRIPTION

Various methods for sensor fabrication assembly are known. However, incorporation of a functionalized sensing surface into a device is challenging. Often, in typical fabrication methods, the functionalized sensing surface can be compromised upon encapsulation due to exposure of the sensing surface to environments used in processing which are non-ambient (e.g. the sensing surface can be subject to pH changes, oxidation, heat damage and damage from ultrasonic welding).

### Sensor Assembly Fabrication

In one embodiment, a method of fabricating a sensor assembly comprises the steps of: providing a sensing surface with an anchor species provided thereon, the anchor species comprising a first functional group; disposing a fluid channel over at least a part of the sensing surface such that fluid can be provided to or removed from the sensing surface via the fluid channel; providing an analyte capture species to the fluid channel, wherein each analyte capture species comprises an analyte capture part and a second functional group configured to react with the first functional group; and exposing at least a portion of the sensing surface covered by the fluid channel to photo radiation so as to cause a photo-initiated reaction between the first functional group and the second functional group to thereby couple the analyte capture species to the anchor species on the sensing surface and form a sensing surface with an analyte capture species thereon.

Embodiments therefore advantageously provide an improved sensor assembly fabrication method which foregoes the disadvantages associated with existing sensor assembly fabrication methods. By disposing a fluid channel over a sensor surface before providing an analyte capture species to the sensor surface, this allows the chemistry of the sensor surface, with the analyte capture species coupled thereto, to remain intact. That is, the capture species has not been exposed to the non-ambient environments used in sensor assembly fabrication steps such as encapsulation in which components such as fluid channels and packaging are incorporated into the device. This reduces failure modes and ultimately increases the performance of the sensor itself.

Moreover, the manufacturing process and device are both more customizable, allowing for easy adaptation of the design of the sensor assembly. That is, a base structure with the anchor species in place and with the fluid channel in place can be used as a framework fora number of different sensors. Such further adaptation need not take place during fabrication, and instead could be carried out locally by a user with a specific need.

The provision of the analyte capture species with complementary reactivity to the chemical functional groups on the anchor species of the sensing surface allows a fast, efficient formation of an analyte capture monolayer on the sensing surface with uniform distribution. The fact that the analyte capture species is provided to the fluid channel following disposal of a fluid channel over the sensing surface also improves the quality of the functional layer formed on the sensing surface. As the analyte capture molecule is coupled to the sensing surface after the disposal of the fluid channel over the sensing surface, the analyte capture molecule is not subject to sensor fabrication processing steps which can occur as fluidics are incorporated into the device. This avoids opportunity for damage to the functional surface, which can occur during the fabrication steps.

The resultant surface functionalization also provides improved sensor functionality. In particular, The reaction between the first and second functional groups using this type of “click chemistry” reaction can result in the formation of uniformly distributed monolayers which is in contrast to the uneven functionalization distribution on the sensing surface commonly achieved through drop-casting methods (e.g. through the CRE). Accordingly, embodiments can provide for much more uniform layers of anchor species to be achieved on the sensing surface for higher accuracy sensing.

In the presence of the analyte capture species which has a functional group with complementary reactivity to the functional group on the anchor species, the radiation exposure can cause a click chemistry reaction to proceed between the complementary groups so that an analyte capture species can be bonded to the selected sensing sites through a click chemistry bridge. The photo-initiation of the click chemistry reaction through the exposure is fast, clean (with very few side products) and selective. The selectivity allows for those portions of the surface which were not exposed to the radiation to be subject to reaction with different analyte capture species (that bind different analytes) in a following step upon repetition of the selective radiation exposure.

In an embodiment, a sensor assembly is obtained or obtainable according to any of the above-mentioned methods.

In one embodiment, a sensor assembly comprises: a sensing surface with a bridging species provided thereon; an analyte capture species coupled to the sensing surface through the bridging species; and a fluid channel disposed over at least a part of the sensing surface, such that fluid can be provided to or removed from the sensing surface via the fluid channel, wherein the bridging species comprises a product of a photo-initiated reaction between a first functional group connected to the sensing surface via an anchor species and a second functional group connected to the analyte capture species.

Embodiments therefore advantageously provide a sensor assembly that has an improved functional layer structure. The surface has a uniformly distributed monolayers, in contrast to the uneven functionalization distribution on the sensing surface commonly achieved through drop-casting methods (e.g. through the CRE), and the chemistry of the sensor surface, with the analyte capture species coupled thereto, remains intact during sensing of analyte molecules as the analyte capture species has not been exposed to the non-ambient environments used in sensor assembly fabrication steps such as encapsulation.

### Sensing Surface and Sensing Mechanism

The method can be used to fabricate or manufacture sensor assemblies which use different sensing mechanisms.

In some embodiments, the sensor assembly is a field effect transistor (FED. A FET uses an electric field to control the conductivity of a channel between a source and drain electrode in a semiconducting material. On binding of the analyte to the analyte capture species on the sensing surface, the charge distribution at the sensing surface is changed and the electrostatic surface potential of the semiconductor is changed. This results in a change of current between the source and drain electrodes and, accordingly, the binding of the analyte can be measured. For example, a FET-biosensor arrangement could be employed to sense biomolecules on a biochemically sensitive surface.

In other embodiments, the sensor assembly comprises at least one working electrode defining the sensing surface. Changes in or interactions with the functional layer formed on the surface can be detected through changes in potential. For example, the molecules adhered to the surface may change position relative to the sensing surface and accordingly change the sensing potential. In other cases, the binding of analyte to the surface may result in a change in potential. Examples of the types of molecule which are commonly employed for this mode of sensing mechanism include aptamers and nucleic acids. These molecules may also be tagged with a redox label such as methylene blue.

In other embodiments, the sensor assembly may comprise a sensing surface through which current is passed (for example, where the sensing surface is a resistive element located between first and second electrodes). This may otherwise function in a similar way to the embodiment comprising the working electrode.

The sensing surface can be a surface of a sensor layer (e.g. an electrode). In some embodiments, this may comprise or be formed from copper, nickel, platinum, silver, silver chloride, gold or other noble metals. In some embodiments, this may comprise or be formed from TiO₂ or indium tin oxide (ITO). Other sensing surfaces may include a substrate with a coating on which the anchor species is immobilized. For example, the sensing surface may be a glass substrate with an ITO coating thereon. In other embodiments, the sensing surface may comprise or be formed of carbon (graphene, graphene oxide, or nanotubes), silicon dioxide, aluminum oxide, and/or silicon.

Sensing surfaces can provide immobilization of anchor species through both covalent-like interactions (e.g. chemisorption of anchor species onto the surface through chemical bond formation) and non-covalent-like interactions (e.g. physisorption of anchor species onto the surface through weaker, often van der Waals, interactions) depending on the identity of the surface and the anchor species.

For example, common sensor surfaces which immobilize anchor species through physisorption are negatively charged surfaces such as metal oxides. These negatively charged metal oxide surfaces can be used to immobilize positively charged species including graft polymers such as polyethylene glycol (PEG). Alternatively, common sensor surfaces which immobilize through chemisorption include gold, silver and copper. Examples of the types of anchor species which bind to these sensing surfaces include silanes (R—Si(OH)₃) and thiols (R—SH).

In another embodiment, the sensing assembly is a resonant mass sensor. Resonant mass sensors work through piezoelectricity—i.e. the ability of a material to produce voltage when mechanically stressed. When a sensor surface is excited by alternating voltage on the surface by two electrodes, this causes mechanical oscillations of the sensor surface. When an analyte is bound to the sensor surface, the frequency of these oscillations is changed proportional to the mass bound to the surface. Accordingly, analyte detection can be carried out wherein specific analytes can change the oscillation on a sensor surface by a specific amount.

In one embodiment, the step of providing a sensing surface with an anchor species provided thereon comprising providing a sensing surface and adhering an anchor species to the sensing surface. Provision of the anchor species to the sensing surface can be achieved through techniques such as spin-coating, physical vapour deposition or electrophoretic deposition. Alternatively, other methods can include immersion of the sensing surface in solution.

Embodiments therefore provide a versatile array of sensing surfaces which allow functionalization to receive analyte capture molecules after the addition of fluidics to avoid damage to the functional layer.

Certain embodiments provide a sensing surface with first and second sensing sites. The first sensing site has the anchor species comprising the first functional group disposed thereon and the second sensing site has a second anchor species comprising a third functional group disposed thereon. The third functional group may be the same as the first functional group or of a different identity. The sensing surface can act as one sensing site or there can be multiple sensing sites on any given sensing surface. For example, where there are multiple sensing sites, there can be greater than 1, greater than 5, or greater than 10 sensing sites on any given sensing surface. For example, there can be 2, 3, 4, 5, 6, 7, 8, 9, 10, 20 or 100 sensing sites. The provision of more than one sensing site on the sensing surface can allow for the detection of multiple different analytes through the immobilization of different analyte capture species with affinities for different analytes on each of the sensing sites on the sensing surface. This allows for multiplexed sensing and fabrication of a multiplexed sensor assembly.

Depending on the sensing surface, different anchor species can be used (as mentioned above). Most commonly used sensing surfaces are gold electrodes in combination with thiol-terminated anchor species. Many anchor species, such as the thiol-terminated types, allow for formation of uniformly distributed self-assembled monolayers on the sensing surface.

Certain embodiments provide anchor species comprising a first functional group. The first functional group can react, upon photo-initiation, with a second functional group on an analyte capture species so as to couple the analyte capture species to the anchor species on the sensing surface.

Certain embodiments provide anchor species with a first functional group on a first sensing site and a second anchor species with a third functional group on a second sensing site. The third functional group may be the same as the first functional group or of a different identity. This allows for multiplexed sensing and fabrication of a multiplexed sensor assembly.

### Analyte Capture Species and Analyte

Analyte capture molecules comprise an analyte capture part and a second functional group. The second functional group is configured to react with the first functional group of the anchor species so as to couple the analyte capture species to the sensing surface via the anchor group as described above. By ‘configured to react with the first functional group’ it is meant that the second functional group is selected based on its chemical identity and ability to react with the first functional group under photo-initiated reaction conditions.

The analyte capture species is coupled to the anchor species directly through reaction of the first functional group on the anchor species and the second functional group on an analyte capture species. When formed, the analyte capture molecule is thus linked to the anchor species via a linker such as a conjugate bridge wherein the second functional group is located on a terminal end of the linker as part of the analyte capture molecule. For example, in some cases a biotin-streptavidin conjugate bridge can link the anchor species to the analyte capture part of the analyte capture species through reaction of the first and second functional groups. Those skilled in the art will appreciate may other types of links which could be formed between the anchor species and the analyte capture part of the analyte capture species.

Any suitable analyte capture species can be selected, according to the analyte which is intended to be sensed by the sensor assembly. For example, the capture species may comprise an antibody with specificity for a particular antigen. In such an example, the analyte may take the form of the antigen. More generally, the capture species may, in some embodiments, comprise at least one selected from a protein, a peptide, a carbohydrate, and a nucleic acid. The protein may, for example, be an enzyme, such as an enzyme having specificity for the analyte. In other non-limiting examples, the protein is an antibody. In the latter case, the analyte may be an antigen which is selectively bound by the antibody. The capture species may, for instance, comprise or be defined by an antigen. In this case, the analyte may be a species, such as an antibody, which is selectively bound by the antigenic capture species. The antigen may be or comprise, for example, a protein, a peptide, a carbohydrate, such as a polysaccharide or glycan.

In an embodiment, the analyte capture species comprises an aptamer. An aptamer may be defined as an oligonucleotide or peptide configured to bind the analyte. Such an aptamer may, for example, be configured to interact with, for example bind, various analyte types, such as small molecules, for example amino acids or amines, proteins, metal ions, and microorganisms. In some non-limiting examples, the aptamer is functionalized with an electro-active moiety, for example a redox-active moiety, and is configured such that a conformational change of the aptamer upon selectively interacting with, for example binding, the analyte causes a change in the proximity of the electro-active moiety with respect to the surface of the respective test electrode. Thus, the aptamer being functionalized with such an electro-active moiety can assist with amperometric sensing of the analyte. The proximity change resulting from the aptamer interacting with, for example binding, the analyte could, for instance, result in the electro-active moiety moving closer to the sensing surface than when the aptamer is not interacting with the analyte. In such examples, electron transfer between the electro-active moiety and the sensing surface may become faster, such as to contribute to an increase in current in an electrode of the sensing surface upon interaction between the analyte and the aptamer. In alternative non-limiting examples, the proximity change resulting from the aptamer interacting with, for example binding, the analyte could result in the electro-active moiety moving further from the sensing surface than when the aptamer is not interacting with the analyte. In such examples, the aptamer may be regarded as being conformationally configured in the absence of the analyte such that the electro-active moiety, for example redox-active moiety, is proximal to, or even in contact with, the sensing surface, thereby providing a baseline signal. In such cases, a decrease in current in the sensing surface upon interaction between the analyte and the aptamer may be observed. Thus, the greater the concentration of analyte, the greater the decrease in the current. Any suitable electro-active moiety may be included in the aptamer for this purpose, such as methylene blue.

The analyte may, for example, be selected from a molecular species, a metal ion, a virus, and a microorganism. Biomarkers, such as a cytokine or a hormone, have relevance in the context of patient monitoring, and diagnostic testing. The analyte may, for instance, be a hormone selected from an eicosanoid, a steroid, an amino acid, amine, peptide or protein.

In one embodiment, the sensing surface comprises first and second sensing sites; and the first sensing site has the anchor species comprising the first functional group disposed thereon; and the second sensing site has a second anchor species comprising a third functional group disposed thereon, wherein the third functional group can be the same as or different to the first functional group. In this way, plural sensing platforms are provided, which can be used for different configurations of sensing sites. For example, this can be used for multiple sensing points or multiplexing. Multiple sensing sites may be part of a single sensing surface, or may be separate sensing surfaces. Each sensing surface may generate a separate signal, which can be used to infer binding on the sensing surface.

Certain embodiments provide for the provision of a second analyte capture species to the fluid channel. In particular, in an embodiment, the method comprises providing a second analyte capture species to the fluid channel, wherein each second analyte capture species comprises: a second analyte capture part with an affinity for a different analyte; and a fourth functional group configured to react with the third functional group on the second anchor species; and exposing at least a portion of the second sensing site to photo radiation such that the third functional group on the anchor species on said portion of the second sensing surface reacts with the fourth functional group on the second analyte capture species so as to couple the anchor species to the second analyte capture species such that a sensor assembly with more than one sensing surface is formed, each sensing surface with a different analyte capture species The fourth functional group on the second analyte capture species may be the same as or different to the second functional group on the first analyte capture species. Accordingly, multiple sensing sites can be provided, each with a different analyte capture species with an affinity for a different analyte provided thereon. This allows for the sensing of multiple types of analyte to occur, also known as multiplexing. The sensing surface can act as one sensing site or there can be multiple sensing sites on any given sensing surface. For example, where there are multiple sensing sites, there can be greater than 1, greater than 5, or greater than 10 sensing sites on any given sensing surface. For example, there can be 2, 3, 4, 5, 6, 7, 8, 9, 10, 20 or 100 sensing sites. The method is particularly advantageous in this respect since different sites can be selectively functionalized using the improved method. Moreover, the process is very straightforward compared to existing fabrication techniques, since the different surfaces can be functionalized simply by directing the light to the site of interest (e.g. using directed light sources or masks). In this way, one capture species can be provided to the fluid channel (which can be in contact with both sensing sites) and only one sensing site (e.g. a portion of a sensing surface or a separate sensing surface) irradiated so as to adhere that capture species to the irradiated sensing site. Subsequently, an additional, different capture species can be provided to the fluid channel and another sensing site irradiated to adhere the different capture species to the other sensing site.

### Reaction Between Functional Groups on Anchor Species and Analyte Capture Species

The reaction between the first or third functional group on the anchor species and the second or fourth functional group on the analyte capture species (either the analyte capture species alone or the analyte capture species with the analyte bound), respectively, takes place after disposal of the fluid channel over the sensing surface. Accordingly, the addition of the second or fourth functional group-containing species can react with the first or third functional group to form the sensing surface.

The reaction of the first or third functional group and the second or fourth functional group, respectively, proceeds via a click chemistry type of reaction in the presence of photo radiation. The reactions are fast and efficient (with no/few side products). Moreover, this provides a customisable fabrication platform, since there are a wide range of options available for the identity of the click chemistry functional groups on both the anchor species and the analyte capture species that can undergo the photo-initiated reaction. The identities of the groups on either the anchor species or the analyte capture molecule species can be interchanged. The click chemistry reactive groups can therefore in some embodiments comprise or consist of, but are not limited to, combinations of the following: azide plus alkyne; thiol plus alkene; tetrazole plus alkene; and Diels Alder cis diene plus alkene reagents.

For example, the first or third functional group (FG1/FG3) can be a thiol, alkene, alkyne, azide, tetrazole, isonitrile, tetrazine, syndone, azirine, enol, epoxide, isocyanate, hydrazone or oxime group. The first or third functional group reacts with a second functional group on the analyte capture species upon photo-initiation. The second or fourth functional group (FG2/FG4) accordingly has complementary reactivity to the first of third functional group and can be any group selected from the same list for which a reaction can occur. For example some examples of the first or third and second or fourth functional group combinations can include: FG1/FG3=thiol and FG2/FG4=alkene; FG1/FG3=azide and FG2/FG4=alkyne; and FG1/FG3=tetrazole and FG2/FG4=alkene.

The photo radiation used to initiate the reaction between the first and second or third or fourth functional groups can be any suitable wavelength. In some embodiments, the method uses UV light as a photo radiation. In some embodiments, the wavelength of photo radiation can range from anywhere between from 300 to 900 nm. For example, the wavelength of photo radiation can be from 320 to 350 nm, from 355 to 370 nm, or from 600 to 900 nm. The wavelength of light photo radiation be, for example, 301 nm, 302 nm, 303 nm, 304 nm, 305 nm, 395 nm or 400 nm. A source can be selected from any photo-radiation source including, but not limited to, a scanning laser beam or LED array. The photo radiation source can be positioned in such a way to form a particular exposure pattern on the sensing surface. For example, this can be achieved by use of a photomask, projection lithography or using an array of addressable LEDs. Different types of radiation source can provide for different wavelengths of light depending upon the wavelength required for the selected first and second functional groups. In one embodiment, the step of exposing at least a portion of the sensing surface to photo radiation comprises at least one of: positioning a photomask to direct photo radiation to a specific part of the sensing surface; using projection lithography; using a scanning laser; or using an array of addressable LEDs.

In some embodiments, heat sources could be introduced to speed up the photo-initiated reaction between first and second functional groups. Without limitation, this heating could be achieved by heat pumps dispersed throughout the fluid channel or external heat sources surrounding the fluid channel.

The provision of the fluid channel over the sensing surface prior to the addition of the analyte capture species to the sensing surface allows the analyte capture species to remain undisturbed by encapsulation and capable of further reactivity to bind an analyte and perform sensing to the highest accuracy.

The fluid channel can be a microfluid channel and can optionally include multiple pumps, valves and other structures along the channel to improve the flow of the reagent across the surface or remove fluid from the surface.

In one embodiment, the fluid channel comprises at least one wall that is at least partially transparent to photo radiation; and the step of exposing at least a portion of the sensing surface covered by the fluid channel to photo radiation comprises directing photo radiation through the at least partially transparent wall. In this way, a photo radiation source can be used externally to the sensor assembly and irradiate the sensing surface to cause the reaction between the functional groups. This can speed up fabrication, avoid the need for more complex manufacturing processes and enable the use of off-the-shelf manufacturing tools.

Certain embodiments provide the fluid channel as a housing provided over the sensing surface. In one embodiment the fluid channel is disposed over the sensing surface. This provides the sensing surface with access to the analyte capture species which are added to the fluid channel prior to the addition of analyte through said channel. In one embodiment, the sensing surface defines a portion of the fluid channel so that the sensing surface is enclosed within the fluid channel and is exposed to fluid passing directly through the fluid channel.

In some embodiments, the fluid channel is an enclosed fluid channel. By enclosed fluid channel it is meant that at least a portion (and in some embodiments all) of the channel is enclosed on all sides, forming a fluid conduit. This can be enclosed by the walls of the fluid channel entirely or by the wall(s) of the fluid channel being provided against another surface, such as a sensing surface.

In some embodiments, prior to the step of providing an analyte capture species to the fluid channel, the method further comprises mixing the analyte capture species with a sample comprising an analyte such that the analyte binds to the analyte capture species via the analyte capture part such that the step of providing an analyte capture species to the fluid channel comprises providing the analyte capture species with the analyte bound thereto to the fluid channel.

The analyte bound to the analyte capture species retains the second functional group on the analyte capture species to enable coupling with the sensing surface via reaction with the first functional group of the anchor species. This provides a particularly advantageous sensing mechanism whereby the connection of the first and second functional groups is only carried out after the binding of the analyte capture species to the analyte. Subsequent irradiation accordingly simultaneously adheres both the analyte capture species and the analyte to the surface, providing the sensing signal. This can speed up the binding reaction times and therefore reduce the overall sensor measurement time.

This allows the photo-initiated binding of the click chemistry reactive groups to be incorporated into the sensing mechanism as opposed to the sensor fabrication. In this way, the analyte is bound to the analyte capture species which is functionalized with a complementary click chemistry reactive group to the anchor species first or third functional group and which binds to the anchor species upon photo radiation exposure.

In some embodiments, the analyte capture species/analytes bound to the analyte capture species may be added in solution to the fluid channel. In other embodiments, the analyte capture species/analytes bound to the analyte capture species may be added in solid form to the fluid channel and react with the first or third functional groups on the anchor species, e.g. through a mechanism similar to stamping/micro contact printing.

### Target Capture Surface

Provision of a target capture surface can be incorporated as a step into the method to allow for capture of various targets, for example from a mixture containing targets and analytes.

In some embodiments, the method of sensor assembly fabrication further comprises the steps of: providing a target capture surface, wherein the target capture surface is provided with an anchor species thereon, the anchor species comprising a fifth functional group; providing a target capture species to the target capture surface, wherein each target capture species comprises a target capture part and a sixth functional group configured to react with the fifth functional group; and exposing at least a portion of the target capture surface to photo radiation so as to cause a photo-initiated reaction between the fifth functional group and the sixth functional group to thereby couple the target capture species to the anchor species on the target capture surface so as to form a target capture surface with a target capture species thereon.

Embodiments therefore advantageously provide a target capture surface as part of the sensor assembly in which the target capture species can be attached to the target capture surface to capture (e.g. filter) targets. This allows for sensing of a fluid containing analytes and target species whereby, for example, the fluid may first be filtered of the target molecules and then the resulting fluid comprising the analytes can be sensed without any target molecule species interference. In some embodiments, the fluid channel comprises the target capture surface.

“Target” as used herein can be the same as the “analyte” set out herein. The term “target” is used to denote that these not necessarily be analysed, although in some embodiments the target may be an analyte. As a result, the target capture species referred to herein is defined in the same way as the analyte capture species set out herein, and may have any of the features defined in respect of the target capture species. Embodiments set out with respect to either apply equally to the other. In some embodiments, the “target” may be the same species as the “analyte”. This can be advantageous in embodiments where the target capture surface is located downstream of the sensing surface. For example, this may be used to capture and retain the analyte after sensing (e.g. for disposal). In other embodiments, the “target” may be a different species to the “analyte”. In this way, the “target” may be filtered out so that a better analyte reading may be taken (e.g. if the target capture surface is upstream of the sensing surface) or for disposal of the target (whether upstream or downstream).

In some embodiments, the target capture surface further comprises a second anchor species disposed thereon, the second anchor species comprising a seventh functional group, the method further comprising: providing a second target capture species to target capture surface, wherein each target capture species comprises: a second target capture part with an affinity for a different target; and an eighth functional group configured to react with the seventh functional group on the second anchor species; and exposing at least a portion of the target capture surface to photo radiation such that the seventh functional group on the anchor species on said portion of the target capture surface reacts with the eighth functional group on the second target capture species so as to couple the anchor species to the second target capture species such the target capture surface is provided with different target capture species thereon.

The anchor species and the second anchor species may be the same as the anchor species provided on the sensing surface. Alternatively, these may be different. Similarly, the anchor species and the second anchor species provided on the target capture surface may be the same as one another or different. In each of these embodiments, the embodiments set out in respect of the anchor species of the sensing surface apply equally to the anchor species of the target capture surface. For example:

In some embodiments, the fifth and/or seventh functional group on the anchor species is selected from a thiol, alkene, alkyne, azide, tetrazole, isonitrile, tetrazine, syndone, azirine, enol, epoxide, isocyanate, hydrazone or oxime group.

In some embodiments, the sixth and/or eighth functional group on the target capture species is selected from a thiol, alkene, alkyne, azide, tetrazole, isonitrile, tetrazine, syndone, azirine, enol, epoxide, isocyanate, hydrazone or oxime group.

The reaction of the fifth or seventh functional group and the sixth or eighth functional group, respectively, proceeds via a click chemistry type of reaction in the presence of photo radiation as for the reaction between the functional groups on the sensing surface with the analyte capture species. The reactions are fast and efficient (with no/few side products). Moreover, this provides a customisable fabrication platform, since there are a wide range of options available for the identity of the click chemistry functional groups on both the anchor species and the target capture species that can undergo the photo-initiated reaction. The identities of the groups on either the anchor species or the target capture molecule species can be interchanged. The click chemistry reactive groups can therefore in some embodiments comprise or consist of, but are not limited to, combinations of the following: azide plus alkyne; thiol plus alkene; tetrazole plus alkene; and Diels Alder cis diene plus alkene reagents.

For example, the fifth or seventh functional group (FG5/FG7) can be a thiol, alkene, alkyne, azide, tetrazole, isonitrile, tetrazine, syndone, azirine, enol, epoxide, isocyanate, hydrazone or oxime group. The fifth or seventh functional group reacts with a sixth or eighth functional group on the target capture species upon photo-initiation. The sixth or eighth functional group (FG6/FG8) accordingly has complementary reactivity to the fifth or seventh functional group and can be any group selected from the same list for which a reaction can occur. For example some examples of the fifth or seventh and sixth or eighth functional group combinations can include: FG5/FG7=thiol and FG6/FG8=alkene; FG5/FG7=azide and FG6/FG8=alkyne; and FG5/FG7=tetrazole and FG6/FG8=alkene.

In some embodiments, the target capture surface comprises the anchor species. In other words, the target capture surface is at least partly formed from a material comprising the anchor species.

In some embodiments, the target capture surface comprises a polymer comprising the anchor species comprising the fifth/seventh functional group. In some embodiments, the polymer is an ostemer (off stoichiometry thiol-ene) polymer. For example, in some embodiments the polymer is an ostemer polymer wherein the anchor species comprises either a thiol or alkene as a first functional group which is provided directly for reaction with the second functional group by the polymer. Example materials are, for example, set out in Carlborg et al., ‘Beyond PDMS: off-stoichiometry thiol—ene (OSTE) based soft lithography for rapid prototyping of microfluidic devices’, Lab Chip, 2011, 11, 3136, which is incorporated herein by reference. In some embodiments, target capture surface comprises a polymer made from monomers comprising an anchor species with an alkyne fifth/seventh functional group such that alkyne functional groups are provided directly by the polymer for reaction with the sixth or eighth functional group.

In some embodiments, the target capture surface is in the fluid channel and is provided to the fluid channel prior to the disposal of the fluid channel over at least a part of the sensing surface. In this way, the target capture surface can be disposed within the fluid channel and thus benefit from lack of exposure to the harsh processing conditions that the sensing assembly fabrication often requires.

In some embodiments, the target capture surface is in the fluid channel and is provided to the fluid channel after the disposal of the fluid channel over at least a part of the sensing surface. In this way, the target capture surface can be disposed external to the fluid channel and filter a fluid of targets prior to the entry of the fluid into the fluid channel and before reaching the sensing surface.

In some embodiments, the target capture surface comprises at least one protrusion or raised structure, said protrusion(s) or raised structure(s) comprising the anchor species or having the anchor species provided thereon. For example, in some embodiments the target capture surface is provided with pillars or ridges extending from the target capture surface into the fluid path. These can be micrometer-sized pillars or ridges (e.g. less than 100 micrometers, such as less than 1 micrometer). This can improve sorting/filtering of the mixture comprising the targets through the increase in surface area provided for anchor species on the target capture surface. Other structural features may also be present on the target capture surface. In some embodiments, the target capture surface is provided with a graduated change in texture along the target capture surface. In some embodiments, the target capture surface is provided with a graduated height change along the target capture surface. The structure of the target capture surface can thus be configured in different ways to enhance the target capture mechanism and improve the flow of the mixture through the channel for access to the target capture surface.

### Sensor Assembly

Sensor assemblies can be provided according to any of the above-mentioned methods for sensor assembly fabrication.

In one embodiment, a sensor assembly comprises: a sensing surface with a bridging species provided thereon; an analyte capture species coupled to the sensing surface through the bridging species; and a fluid channel disposed over at least a part of the sensing surface, such that fluid can be provided to the sensing surface via the fluid channel, wherein the bridging species comprises a product of a photo-initiated reaction between a first functional group connected to the sensing surface via an anchor species and a second functional group connected to the analyte capture species.

The “bridging species” is defined as the product of reaction between the functional group on the anchor species and the functional group on the analyte capture species following a photo-initiated reaction. For example, this could be the product of a photo-initiated reaction between a thiol and an alkene which includes a bond formed between the sulphur of the thiol and the vinylic carbon of the alkene (in such a thiol-alkene example). Other examples of bridging species can include, but are not limited to, various bonds formed between different complementary functional groups on the anchor species and analyte capture species (e.g. the bond formed between the alkyne carbons on an alkyne functional group and the nitrogens on an azide functional group). In this way the bridging species can be seen as a tether on the sensing surface.

In an embodiment, a sensor assembly comprises a sensing surface with first and second sensing sites (in some embodiments, plural sensing sites), each sensing site having a bridging species disposed thereon. In some embodiments, the first sensing site has a first analyte capture species disposed thereon and the second sensing site has a second analyte capture species disposed thereon. In other words, each sensing site is provided with a different analyte capture species linked to the anchor species via the bridging species. Each analyte capture species on any sensing site can have an affinity for a different analyte so as to allow sensing of different analytes in the same sensor assembly—i.e. the sensor assembly can allow for efficient multiplexation. The sensing surface can act as one sensing site or there can be multiple sensing sites on any given sensing surface. For example, where there are multiple sensing sites, there can be greater than 1, greater than 5, or greater than 10 sensing sites on any given sensing surface. For example, there can be 2, 3, 4, 5, 6, 7, 8, 9, 10, 20 or 100 sensing sites.

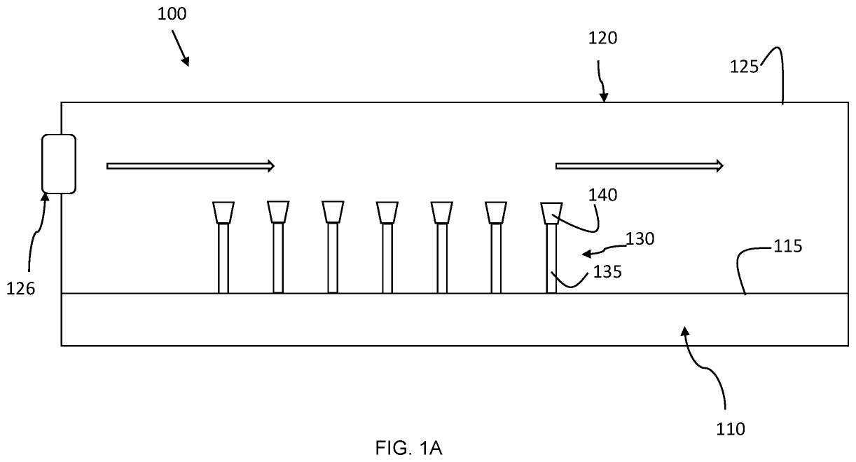

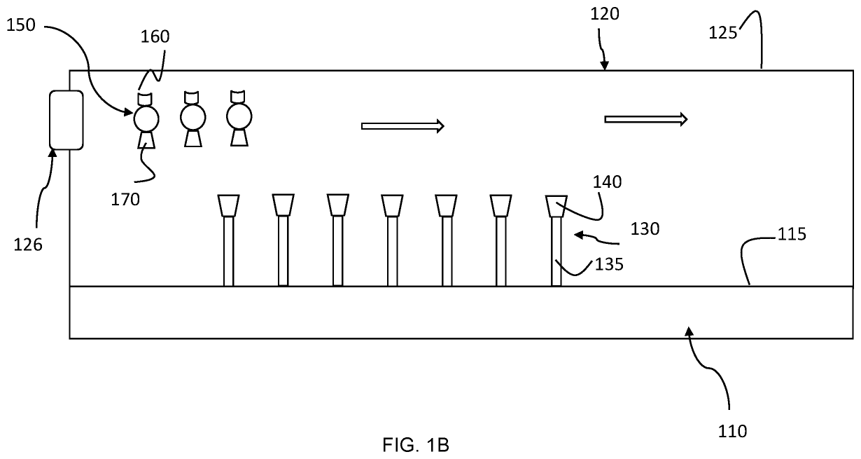

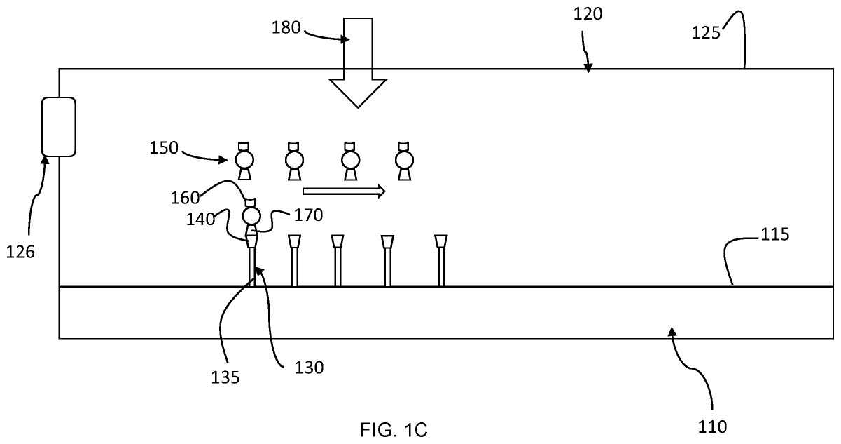

A first embodiment of the method of fabricating a sensing assembly 100 is shown schematically in FIGS. 1A-1C. FIG. 1A shows a sensing surface 110 which is an elongate strip with an upper face 115. The upper face 115 is provided with an anchor species 130 disposed thereon. The anchor species 130 comprises a tail portion 135 and a first functional group 140. The anchor species 130 is adhered to the sensing surface via the tail portion 135 which is attached to the upper face 115 of the sensing surface 110. The first functional group 140 is connected to the tail portion 135 at an upper terminal end of the tail portion 135 and is directed away from the sensing surface 110. A fluid channel 120 is disposed over the entirety of the sensing surface 110. The fluid channel 120 comprises a wall 125 which extends laterally above the anchor species 130 disposed on the sensing surface 110 and which connects to the sensing surface 110 by vertically extending from each side of the lateral extension and thus the fluid channel 120 is configured as a housing over the sensing surface 110. The fluid channel further comprises an inlet 126 to allow the addition of reagents into the fluid channel 120. The arrows indicate the direction of flow of a fluid through the fluid channel 120 and for delivery of reagents to the sensing surface 110.

FIG. 1B shows the step of providing an analyte capture species 150 to sensing surface 110 via the fluid channel 120. The analyte capture species 150 are added to the fluid channel 120 through the inlet 126 so that, after addition, each analyte capture species 150 is within the fluid channel 120, located between the wall 125 and the sensing surface 110. Each analyte capture species 150 comprises an analyte capture part 160 and a second functional group 170 configured to react with the first functional group 140 on anchor species 130. In this embodiment, the analyte capture species 150 is added to the fluid channel 120 in the form of a solution through the inlet 126. The solution accordingly flows through the fluid channel 120 so that the second functional group 170 on the analyte capture species 150 is ready to react with the first functional group 140 on the anchor species 130.

FIG. 1C schematically depicts the step of the binding of the analyte capture species 150 to the sensing surface 110. This step occurs during exposure of the sensing surface 110 to photo radiation 180. The photo radiation 180 enters the fluid channel 120 through the lateral extension of the wall 125 in a position which is directly above the anchor species 130 disposed on the sensing surface 110 so that the anchor species 130 can be targeted by the photo radiation 180. The first functional group 140 of the anchor species 130 reacts with the second functional group 170 of the analyte capture species 150 on exposure to photo radiation 180. Accordingly, the analyte capture part 160 of the analyte capture species 150 adheres to the sensing surface 110 and is therefore ready to sense (capture) an analyte (through binding said analyte at the analyte capture part 160).

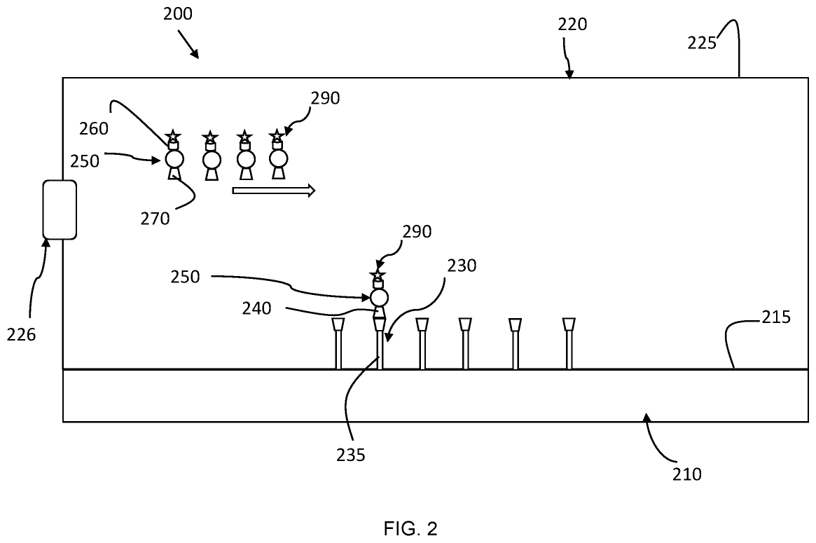

A second embodiment of the method of fabricating a sensing assembly 200 is shown in FIG. 2. FIG. 2 shows a sensing surface 210 comprising an upper face 215 with an anchor species 230 adhered thereto in an identical manner to that as described in the previous embodiment. The anchor species comprises a first functional group 240 and a tail portion 235. The fluid channel 220 comprises a wall 225 and is disposed over the sensing surface 210 in the same manner as for the previous embodiment. FIG. 2 shows the analyte capture species 250, which comprises the analyte capture part 260, second functional group 270 and analyte 290, within the fluid channel 220 after addition through the inlet 226. The method is the same as that depicted schematically in FIGS. 1A to C, except that prior to the step of providing an analyte capture species 250 to the fluid channel 220, the method further comprises mixing the analyte capture species 250 with an analyte 290 so that the analyte 290 is bound to the analyte capture species 250. The analyte capture species 250 with the analyte 290 bound is added to the fluid channel 220 through inlet 226, and the sensing surface 210 is exposed to photo radiation. In this way, the reaction between the first functional group 240 on anchor species 230 and second functional group 270 on analyte capture species 250—i.e. coupling of the analyte capture species 250 to the sensing surface 210 is also a mode of sensing the analyte 290. This speeds up the binding reaction times of the analyte capture species 250 to the sensing surface 210 and the binding of the analyte 290 to result in a quicker overall sensing measurement time. Differentiation between a capture species 250 which is unbound and a capture species 250 to which an analyte 290 is bound can be achieved based on the response of the signal from the sensor assembly.

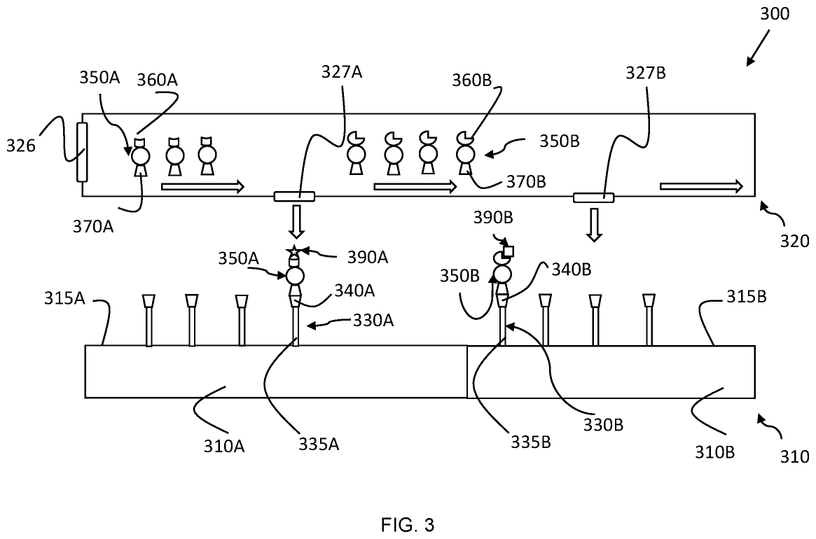

Another embodiment of the method of fabricating a sensing assembly 300 is shown in FIG. 3. FIG. 3 shows a sensing surface 310 which is an elongate strip comprising two sensing sites 310A and 310B as two connected portions which make up the whole sensing surface 310. Each sensing site has a respective upper face portion 315A, 315B. Upper face portion 315A has an anchor species 330A disposed thereon and upper surface portion 315B has an anchor species 330B disposed thereon. Each anchor species 330A, 330B comprises a respective tail portion 335A, 335B and a functional group. Anchor species 330A comprises a first functional group 340A configured to react with a second functional group 370A of a first analyte capture species 350A and anchor species 330B comprises a third functional group 340B configured to react with a fourth functional group 370B of a second analyte capture species 350B. Each anchor species 330A, 330B is immobilized on the respective upper face portion 315A, 315B of the sensing site through a respective tail portion 335A, 335B. FIG. 3 further shows a fluid channel 320 disposed over the entirety of the sensing surface 310. The fluid channel 320 is disposed over the sensing surface 310 in a way that can be achieved through addition of a layer on top of the sensing surface into which the fluid channel 320 is incorporated. The fluid channel 320 comprises an inlet 326 for addition of reagents into the fluid channel 320 and outlets 327A, 327B for addition of the reagents to access the sensing surface 310. A first analyte capture species 350A is added to the fluid channel 320 through inlet 326, in addition to a second analyte capture species 350B. The second analyte capture species 350B has an affinity for a different analyte 390B compared to the first analyte capture species 350A with affinity for analyte 390A. First sensing site 310A is exposed to photo radiation separately to second sensing site 310B. Accordingly, on exposure to the photo radiation, the first functional group 340A on the anchor species 330A reacts with the second functional group 370A on the first analyte capture species 350A. Separately, on exposure to photo radiation, the third functional group 340B on the anchor species 330B reacts with the fourth functional group 370B on the second analyte capture species 350B. Accordingly, the sensing surface 310 comprises first sensing site 310A coupled to first analyte capture species 350A which has an affinity for analyte 390A and second sensing site 310B is coupled to second analyte capture species 350B which has an affinity for analyte 390B. Multiple sensing sites can be provided; however, only one further sensing site is shown for ease of illustration. Multiple further analyte capture species can therefore be added to the fluid channel however only one further analyte capture species is shown for ease of illustration. Each further analyte capture species has an affinity for a different analyte.

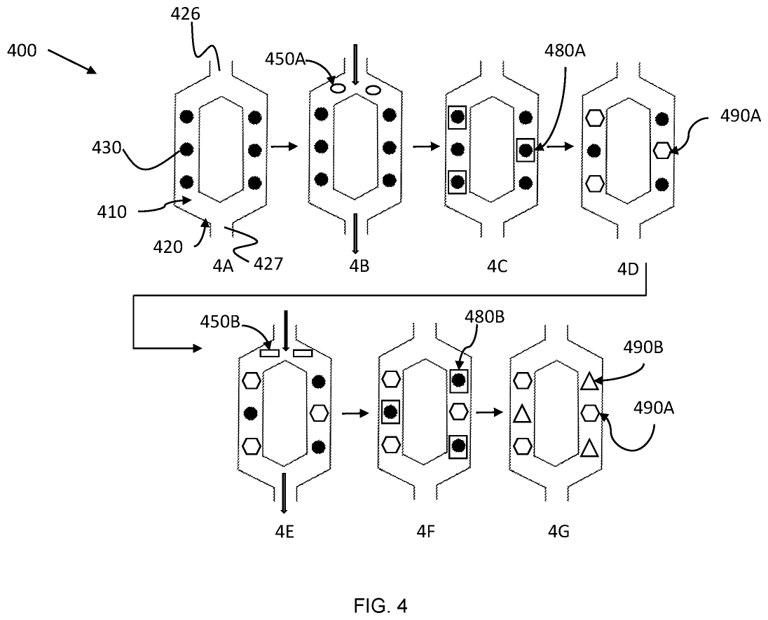

A further embodiment of the method of fabricating a sensing assembly 400 is shown in FIG. 4, panels 4A-4G. FIG. 4, panel 4A shows a sensing surface 410 disposed within a fluid channel 420. The fluid channel 420 is configured as two pathways which branch from an initial inlet 426 and which lead to an outlet 427 where the two pathways recombine. The sensing surface 410 comprises anchor species 430. The sensing surface 410 is disposed along each pathway so that anchor species 430 are disposed on each pathway. As for the previous embodiments, each anchor species 430 comprises a tail portion and a first functional group (not shown) and the anchor species 430 is immobilized on the sensing surface 410 through the tail portion. FIG. 4, panel 4B shows analyte capture species 450A added to fluid channel 420. As for previous embodiments, analyte capture species 450A comprises an analyte capture part and a second functional group configured to react with the first functional group of the anchor species (not shown). FIG. 4, panel 4C shows, in a next step, the anchor species 430 is selectively exposed to photo radiation through a photomask to form selected anchor species 480A with an analyte capture species 450A bound thereto. Whilst not shown, the photomask is directly above the fluid channel 420 and is configured to mask sites which are not intended to bind an analyte capture species 450A. FIG. 4, panel 4D shows that, in a next step, an analyte is added to the fluid channel and binds to the analyte capture species 450A to form an anchor species with an analyte bound thereto 490A via the analyte capture species. Accordingly, the sensing surface 410 comprises anchor species 430 which remains unbound and anchor species with an analyte bound thereto 490A. FIG. 4, panel 4E shows the continuation of the method of fabrication with the addition of a different analyte capture species 450B to the fluid channel 420. FIG. 4, panel 4F shows that the remaining unbound anchor species 430 are selectively exposed to photo radiation through a photomask to from selected anchor species 480B with an analyte capture species 450B bound thereto. FIG. 4, panel 4G shows, in a next step, a different analyte with an affinity for analyte capture species 450B is added to the fluid channel 420 to form an anchor species with an analyte bound thereto 490B via the analyte capture species. Accordingly, FIG. 4, panel 4G shows the sensing surface 410 comprising two different types of analyte bound to anchor species, 490A and 490B. Multiple further analyte capture species can be added to the fluid channel however only one further analyte capture species is shown for ease of illustration. Each further analyte capture species has an affinity for a different analyte.

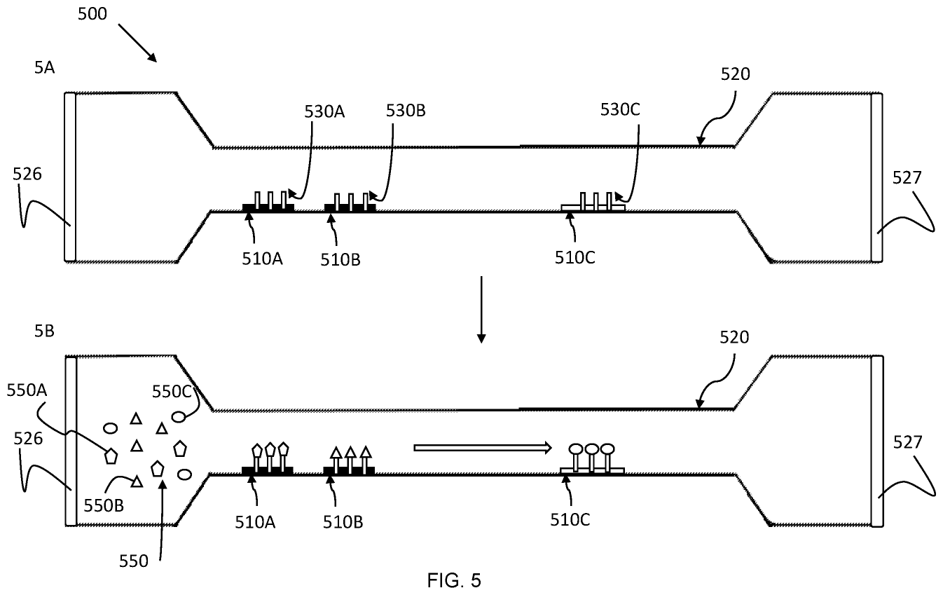

A further embodiment of the method of fabricating a sensing assembly 500 is shown in FIG. 5, panels 5A-5B. FIG. 5, panel 5A shows fluid channel 520 provided as a conduit with an inlet 526 and outlet 527. The fluid channel comprises target capture surfaces 510A and 510B. The target capture surfaces 510A and 510B are provided within the fluid channel 520 and are respectively provided with target capture species 530A and 530B bound thereto. The target capture species are provided on the target capture surface following reaction of a fifth functional group on an anchor species with a sixth functional group on a target capture species in the presence of photo radiation (not shown). The fluid channel 520 further comprises sensing surface 510C. The sensing surface 510C can be any sensing surface as described in accordance with any of the previous embodiments. The sensing surface is disposed within the fluid channel along the path of fluid flow after the target capture surfaces 510A and 510B. The sensing surface 510C comprises an analyte capture species bound to an anchor species 530C disposed on the sensing surface 510C. FIG. 5, panel 5B shows the addition of a mixture 550 comprising targets 550A and 550B in addition to analytes 550C to the fluid channel 520. The fluid flows through the fluid channel 520 as indicated by the direction of the arrow within the fluid channel 520. As the mixture 550 flows through the fluid channel 520, the targets 550A and 550B are captured by the respective target capture species 530A and 530B bound to the respective surfaces 510A and 5108. The remaining analytes 550C in the mixture 550 are then able to flow over the sensing surface 510C for detection. Accordingly, the mixture 550 is filtered leaving the analytes 550C for detection over the sensing surface 510C.

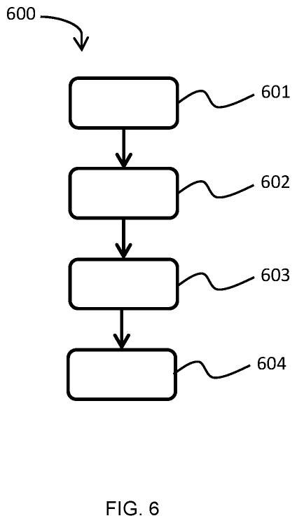

FIG. 6 provides a block diagram of a method of sensor assembly fabrication 600 according to an example. The step of providing a sensor surface with an anchor species provided thereon, the anchor species comprising a first functional group is represented by 601; disposing a fluid channel over at least a part of the sensing surface such that fluid can be provided to or removed from the sensing surface via the fluid channel by 602; providing an analyte capture species to the fluid channel, wherein each analyte capture species comprises an analyte capture part and a second functional group configured to react with the first functional group by 603; and exposing at least a portion of the sensing surface covered by the fluid channel to photo radiation so as to cause a photo-initiated reaction between the first functional group and the second functional group to thereby couple the analyte capture species to the anchor species on the sensing surface and form a sensing surface with an analyte capture species thereon by 604.

### EXAMPLES

Examples of specific capture species and functional groups will now be set out.

In an example, a method of fabrication of a sensor assembly comprises providing a SiO₂ sensing surface with an anchor species disposed thereon. The anchor species comprises a thiol group as a first functional group disposed thereon. A fluid channel is disposed over the sensing surface. The fluid channel is filled with an analyte capture species which is an alkene-terminated biotinylated species. The alkene is the second functional group and the analyte capture species has an affinity specific to analyte A. The sensing surface is selectively exposed to UV radiation through a photomask so that a reaction between the thiol first functional group and the alkene second functional group occurs forming a bond between the sulphur of the thiol and the vinylic carbon of the alkene. The sensing surface therefore comprises an analyte capture species attached to the anchor species through a bridging species formed by reaction of the thiol and alkene functional groups.

In another example, a method of fabrication of a sensor assembly comprises providing a sensing surface comprising first and second sensing sites. The first sensing site comprises an anchor species comprising a thiol group as the first functional group disposed thereon. The second sensing site comprises an anchor species comprising a thiol group as the third functional group. A fluid channel is disposed over the sensing surface. The fluid channel is filled with a first analyte capture species which is an alkene-terminated biotinylated aptamer wherein the alkene is the second functional group and the analyte capture species has an affinity specific to analyte A. The sensing surface is selectively exposed to UV radiation through a photomask so that the first sensing site is targeted and a reaction between the thiol first functional group and the alkene second functional group occurs forming a bond between the sulphur of the thiol and the vinylic carbon of the alkene. The first sensing site on the sensing surface therefore comprises an analyte capture species attached to the anchor species on the first sensing site through a bridging species formed by reaction of the thiol and alkene functional groups. A second analyte capture molecule is then added to the fluid channel which is an alkene-terminated biotinylated aptamer specific to analyte B. The sensing surface is selectively exposed to UV radiation through a photomask so that the second sensing site is targeted. The second sensing site on the sensing surface therefore comprises an analyte capture species attached to the anchor species on the second sensing site through a bridging species formed by reaction of the thiol and alkene functional groups. Accordingly, the sensing surface comprises two different analyte capture species so that two different corresponding analytes can be sensed.

In an example, a sensor assembly is provided comprising target capture surface, at least a part of which is made out of ostemer polymer. The ostemer polymer comprises excess thiol groups on the surface. A medium comprising a target capture species is added to the fluid channel. The fluid channel is exposed to UV photo radiation and target capture species with a complementary functional group to the thiol group are subsequently bonded to the thiol groups. For example, target capture species with alkene functionalization are added and are bonded to the thiol groups on the surface using photo radiation and a photo ‘click’ reaction.

In an example, a sensor assembly is provided comprising a target capture surface with an anchor species disposed thereon. The anchor species comprises thiol first functional groups disposed thereon. A medium comprising a target capture species is added to the fluid channel. The fluid channel is exposed to UV photo radiation and an alkene-terminated target capture species is subsequently bonded to the thiol group. The sensing surface is an SiO₂ surface comprising thiol-terminated anchor species. Alkene-terminated biotinylated analyte capture species are added to the fluid channel following the step of binding the target capture species to the anchor species on the inner surface. The fluid channel is then selectively exposed to UV photo radiation so as to bind the analyte capture species to the sensing surface via the anchor species. A mixture comprising targets and analytes A is then added to the fluid channel. The mixture flows over the inner surface first and the targets are subsequently bound to the target capture species on the inner surface. The mixture is accordingly filtered of the targets and the analytes A then bind to the analyte capture species on the sensing surface for detection.

Following the binding of the target capture species to the target capture surface, a mixture comprising targets and analytes A is added to the fluid channel. The mixture flows over the target capture surface first and the targets are subsequently bound to the target capture species. The mixture is accordingly filtered of the targets and the analytes A are free to be detected on the sensing surface.

In any of the above examples, the alkene-terminated biotinylated species may, for example be an aptamer or an antibody and the analytes A and B may be selected from COVID-19, MERS, Influenza A or Influenza B. For example, an alkene-terminated biotinylated aptamer can be used which is specific to any of COVID-19, MERS, Influenza A or Influenza B. Likewise, an alkene-terminated biotinylated antibody may be used which is specific to any of COVID-19, MERS, Influenza A or Influenza B.

Example materials of the sensing surface and specific alkene-terminated biotinylated species are, for example, set out in Waldmann et al., ‘Preparation of Biomolecule Microstructures and Microarrays by Thiol-ene Photoimmobilization’, ChemBioChem, 2010, 11, 235, which is incorporated herein by reference.

It should be understood that the detailed description and specific examples, while indicating exemplary embodiments of the apparatus, systems and methods, are intended for purposes of illustration only and are not intended to limit the scope. These and other features, aspects, and advantages of the apparatus, systems and methods of the present invention can be better understood from the description, appended claims or aspects, and accompanying drawings. It should be understood that the Figures are merely schematic and are not drawn to scale. It should also be understood that the same reference numerals are used throughout the figures to indicate the same or similar parts.

Other variations to the disclosed embodiments can be understood and effected by those skilled in the art in practicing the disclosure, from a study of the drawings, the disclosure, and the appended aspects or claims. In the aspects or claims, the word “comprising” does not exclude other elements or steps, and the indefinite article “a” or “an” does not exclude a plurality. The mere fact that certain measures are recited in mutually different dependent aspects or claims does not indicate that a combination of these measures cannot be used to advantage. Any reference signs in the claims should not be construed as limiting the scope.

In a first aspect, a method of fabricating a sensor assembly comprises the steps of:
providing a sensing surface with an anchor species provided thereon, the anchor species comprising a first functional group;
disposing a fluid channel over at least a part of the sensing surface such that fluid can be provided to or removed from the sensing surface via the fluid channel;
providing an analyte capture species to the fluid channel, wherein each analyte capture species comprises an analyte capture part and a second functional group configured to react with the first functional group; and
exposing at least a portion of the sensing surface covered by the fluid channel to photo radiation so as to cause a photo-initiated reaction between the first functional group and the second functional group to thereby couple the analyte capture species to the anchor species on the sensing surface and form a sensing surface with an analyte capture species thereon.

Aspect 2. The method of fabricating a sensor assembly according to aspect 1, wherein prior to the step of providing an analyte capture species to the fluid channel, the method further comprises mixing the analyte capture species with a sample comprising an analyte such that the analyte binds to the analyte capture species via the analyte capture part such that the step of providing an analyte capture species to the fluid channel comprises providing the analyte capture species with the analyte bound thereto to the fluid channel.

Aspect 3. The method of fabricating a sensor assembly according to any preceding aspect, wherein the first functional group on the anchor species is selected from a thiol, alkene, alkyne, azide, tetrazole, isonitrile, tetrazine, syndone, azirine, enol, epoxide, isocyanate, hydrazone or oxime group.

Aspect 4. The method of fabricating a sensor assembly according to any preceding aspect, wherein the second functional group on the analyte capture species is selected from a thiol, alkene, alkyne, azide, tetrazole, isonitrile, tetrazine, syndone, azirine, enol, epoxide, isocyanate, hydrazone or oxime group.

Aspect 5. The method of fabricating a sensor assembly according to any preceding aspect, wherein the step of exposing at least a portion of the sensing surface to photo radiation comprises at least one of: positioning a photomask to direct photo radiation to a specific part of the sensing surface; projecting a pattern onto the sensing surface using projection lithography; using a scanning laser; or using an array of addressable LEDs.

Aspect 6. The method of fabricating a sensor assembly according to any preceding aspect, wherein:
the sensing surface comprises first and second sensing sites;
wherein the first sensing site has the anchor species comprising the first functional group disposed thereon; and
the second sensing site has a second anchor species comprising a third functional group disposed thereon, wherein the third functional group can be the same as or different to the first functional group.

Aspect 7. The method of fabricating a sensor assembly according to any preceding aspect, further comprising:
providing a second analyte capture species to the fluid channel, wherein each second analyte capture species comprises: a second analyte capture part with an affinity for a different analyte; and a fourth functional group configured to react with the third functional group on the second anchor species; and
exposing at least a portion of the second sensing site to photo radiation such that the third functional group on the anchor species on said portion of the second sensing surface reacts with the fourth functional group on the second analyte capture species so as to couple the anchor species to the second analyte capture species such that a sensor assembly with more than one sensing surface is formed, each sensing surface with a different analyte capture species thereon.

Aspect 8. The method of fabricating a sensor assembly according to any preceding aspect, wherein the fourth functional group is the same as the second functional group.

Aspect 9. The method of fabricating a sensor assembly according to any preceding aspect, wherein the fluid channel comprises at least one wall that is at least partially transparent to photo radiation; and the step of exposing at least a portion of the sensing surface covered by the fluid channel to photo radiation comprises directing photo radiation through the at least partially transparent wall.

Aspect 10. The method of fabricating a sensor assembly according to any preceding aspect, wherein the step of providing a sensing surface with an anchor species provided thereon comprising providing a sensing surface and adhering an anchor species to the sensing surface.

Aspect 11. The method of fabricating a sensor assembly according to any preceding aspect, further comprising the steps of:
providing a target capture surface, wherein the target capture surface is provided with an anchor species thereon, the anchor species comprising a fifth functional group;
providing a target capture species to the target capture surface, wherein each target capture species comprises a target capture part and a sixth functional group configured to react with the fifth functional group; and
exposing at least a portion of the target capture surface to photo radiation so as to cause a photo-initiated reaction between the fifth functional group and the sixth functional group to thereby couple the target capture species to the anchor species on the target capture surface so as to form a target capture surface with a target capture species thereon.

Aspect 12. The method of fabricating a sensor assembly according to aspect 11, wherein the target capture surface further comprises a second anchor species disposed thereon, the second anchor species comprising a seventh functional group, the method further comprising:
providing a second target capture species to target capture surface, wherein each target capture species comprises: a second target capture part with an affinity for a different target; and an eighth functional group configured to react with the seventh functional group on the second anchor species; and
exposing at least a portion of the target capture surface to photo radiation such that the seventh functional group on the anchor species on said portion of the target capture surface reacts with the eighth functional group on the second target capture species so as to couple the anchor species to the second target capture species such the target capture surface is provided with different target capture species thereon.

Aspect 13. The method of fabricating a fabricating a sensor assembly according to any of aspects 11 to 12, wherein the fifth functional group on the anchor species is selected from a thiol, alkene, alkyne, azide, tetrazole, isonitrile, tetrazine, syndone, azirine, enol, epoxide, isocyanate, hydrazone or oxime group.

Aspect 14. The method of fabricating a sensor assembly according to any of aspects 11 to 13, wherein the sixth functional group on the target capture species is selected from a thiol, alkene, alkyne, azide, tetrazole, isonitrile, tetrazine, syndone, azirine, enol, epoxide, isocyanate, hydrazone or oxime group.

Aspect 15. The method of fabricating a sensor assembly according to any of aspects 11 to 14, wherein the fluid channel comprises the target capture surface.

Aspect 16. The method of fabricating a sensor assembly according to aspect 15, wherein the target capture surface comprises the anchor species.

Aspect 17. A sensor assembly obtained or obtainable by the method according to any preceding aspect.

Aspect 18. A sensor assembly comprising:
a sensing surface with a bridging species provided thereon;
an analyte capture species coupled to the sensing surface through the bridging species; and
a fluid channel disposed over at least a part of the sensing surface, such that fluid can be provided to or removed from the sensing surface via the fluid channel,
wherein the bridging species comprises a product of a photo-initiated reaction between a first functional group connected to the sensing surface via an anchor species and a second functional group connected to the analyte capture species.

Aspect 19. A sensor assembly according to aspect 18, wherein the sensing surface comprises first and second sensing sites, each sensing site with a bridging species disposed thereon.

Aspect 20. A sensor assembly according to aspect 18 or aspect 19, wherein the first sensing site has a first analyte capture species coupled thereto and the second sensing site has a second analyte capture species coupled thereto, each analyte capture species having an affinity for a different analyte.

What is claimed is:

1. A method of fabricating a sensor assembly comprising the steps of:
providing a sensing surface with an anchor species provided thereon, the anchor species comprising a first functional group;
disposing a fluid channel over at least a part of the sensing surface such that fluid can be provided to or removed from the sensing surface via the fluid channel;
providing an analyte capture species to the fluid channel, wherein each analyte capture species comprises an analyte capture part and a second functional group configured to react with the first functional group; and
exposing at least a portion of the sensing surface covered by the fluid channel to photo radiation so as to cause a photo-initiated reaction between the first functional group and the second functional group to thereby couple the analyte capture species to the anchor species on the sensing surface and form a sensing surface with an analyte capture species thereon.

2. The method of fabricating a sensor assembly according to claim 1, wherein prior to the step of providing an analyte capture species to the fluid channel, the method further comprises mixing the analyte capture species with a sample comprising an analyte such that the analyte binds to the analyte capture species via the analyte capture part such that the step of providing an analyte capture species to the fluid channel comprises providing the analyte capture species with the analyte bound thereto to the fluid channel.

3. The method of fabricating a sensor assembly according to claim 1, wherein the first functional group on the anchor species is selected from a thiol, alkene, alkyne, azide, tetrazole, isonitrile, tetrazine, syndone, azirine, enol, epoxide, isocyanate, hydrazone or oxime group.

4. The method of fabricating a sensor assembly according to claim 1, wherein the second functional group on the analyte capture species is selected from a thiol, alkene, alkyne, azide, tetrazole, isonitrile, tetrazine, syndone, azirine, enol, epoxide, isocyanate, hydrazone or oxime group.

5. The method of fabricating a sensor assembly according to claim 1, wherein the step of exposing at least a portion of the sensing surface to photo radiation comprises at least one of: positioning a photomask to direct photo radiation to a specific part of the sensing surface; projecting a pattern onto the sensing surface using projection lithography; using a scanning laser; or using an array of addressable LEDs.

6. The method of fabricating a sensor assembly according to claim 1, wherein:
the sensing surface comprises first and second sensing sites;
wherein the first sensing site has the anchor species comprising the first functional group disposed thereon; and
the second sensing site has a second anchor species comprising a third functional group disposed thereon, wherein the third functional group can be the same as or different to the first functional group.

7. The method of fabricating a sensor assembly according to claim 6, further comprising:
providing a second analyte capture species to the fluid channel, wherein each second analyte capture species comprises: a second analyte capture part with an affinity for a different analyte; and a fourth functional group configured to react with the third functional group on the second anchor species; and
exposing at least a portion of the second sensing site to photo radiation such that the third functional group on the anchor species on said portion of the second sensing surface reacts with the fourth functional group on the second analyte capture species so as to couple the anchor species to the second analyte capture species such that a sensor assembly with more than one sensing surface is formed, each sensing surface with a different analyte capture species thereon.

8. The method of fabricating a sensor assembly according to claim 7, wherein the fourth functional group is the same as the second functional group.

9. The method of fabricating a sensor assembly according to claim 1, wherein the fluid channel comprises at least one wall that is at least partially transparent to photo radiation; and the step of exposing at least a portion of the sensing surface covered by the fluid channel to photo radiation comprises directing photo radiation through the at least partially transparent wall.

10. The method of fabricating a sensor assembly according to claim 1, wherein the step of providing a sensing surface with an anchor species provided thereon comprising providing a sensing surface and adhering an anchor species to the sensing surface.

11. The method of fabricating a sensor assembly according to claim 1, further comprising the steps of:
providing a target capture surface, wherein the target capture surface is provided with an anchor species thereon, the anchor species comprising a fifth functional group;
providing a target capture species to the target capture surface, wherein each target capture species comprises a target capture part and a sixth functional group configured to react with the fifth functional group; and
exposing at least a portion of the target capture surface to photo radiation so as to cause a photo-initiated reaction between the fifth functional group and the sixth functional group to thereby couple the target capture species to the anchor species on the target capture surface so as to form a target capture surface with a target capture species thereon.

12. The method of fabricating a sensor assembly according to claim 11, wherein the target capture surface further comprises a second anchor species disposed thereon, the second anchor species comprising a seventh functional group, the method further comprising:
providing a second target capture species to target capture surface, wherein each target capture species comprises: a second target capture part with an affinity for a different target; and an eighth functional group configured to react with the seventh functional group on the second anchor species; and
exposing at least a portion of the target capture surface to photo radiation such that the seventh functional group on the anchor species on said portion of the target capture surface reacts with the eighth functional group on the second target capture species so as to couple the anchor species to the second target capture species such the target capture surface is provided with different target capture species thereon.

13. The method of fabricating a fabricating a sensor assembly according to claim 11, wherein the fifth functional group on the anchor species is selected from a thiol, alkene, alkyne, azide, tetrazole, isonitrile, tetrazine, syndone, azirine, enol, epoxide, isocyanate, hydrazone or oxime group.

14. The method of fabricating a sensor assembly according to claim 11, wherein the sixth functional group on the target capture species is selected from a thiol, alkene, alkyne, azide, tetrazole, isonitrile, tetrazine, syndone, azirine, enol, epoxide, isocyanate, hydrazone or oxime group.

15. The method of fabricating a sensor assembly according to claim 11, wherein the fluid channel comprises the target capture surface.

16. The method of fabricating a sensor assembly according to claim 15, wherein the target capture surface comprises the anchor species.

17. A sensor assembly obtained or obtainable by the method according to claim 1.

18. A sensor assembly comprising:
a sensing surface with a bridging species provided thereon;
an analyte capture species coupled to the sensing surface through the bridging species; and
a fluid channel disposed over at least a part of the sensing surface, such that fluid can be provided to or removed from the sensing surface via the fluid channel,
wherein the bridging species comprises a product of a photo-initiated reaction between a first functional group connected to the sensing surface via an anchor species and a second functional group connected to the analyte capture species.

19. A sensor assembly according to claim 18, wherein the sensing surface comprises first and second sensing sites, each sensing site with a bridging species disposed thereon.

20. A sensor assembly according to claim 19, wherein the first sensing site has a first analyte capture species coupled thereto and the second sensing site has a second analyte capture species coupled thereto, each analyte capture species having an affinity for a different analyte.
